(19) United States
(12) Patent Application Publication
(10) Pub. No.: US 2021/0283295 A1
(43) Pub. Date: Sep. 16, 2021

(54) FLAME LAMP HAVING FRAGRANCE

(71) Applicant: TEST RITE INTERNATIONAL CO., LTD., TAIPEI CITY (TW)

(72) Inventor: YEN-LING HSU, TAIPEI CITY (TW)

(21) Appl. No.: 16/845,527

(22) Filed: Apr. 10, 2020

(30) Foreign Application Priority Data
Mar. 12, 2020 (CN) 202020307265.7

(51) Int. Cl. A61L 9/03; F21S 10/04

(52) U.S. Cl. CPC A61L 9/03; F21Y 2115/10; A61L 2209/12; F21S 10/04

(57) ABSTRACT

The invention provides a flame lamp, which comprises a top cover, a base, a lamp cover, and a control circuit. A tray is embedded in an upper surface of the top cover, and used for holding an essential oil. The control circuit comprises a control board having a microprocessor, a flexible circuit board, and a heater. The flexible circuit board comprises a PWM driver and a plurality of LED lamp beads. When the flame lamp operates, the microprocessor outputs a timing signal to the PWM driver. The PWM driver generates a PWM control signal according to the timing signal, and controls a blinking and light emitting intervals of the LED lamp beads via the PWM control signal, such that a flame effect can be simulated. Besides, the heater is disposed below the tray, and heats the essential oil in the tray to evaporate a fragrance.

This non-provisional application claims priority claim under 35 U.S.C. § 119 (a) on China Patent Application No. 202020307265.7 filed Mar. 12, 2020, the entire contents of which are incorporated herein by reference.

### FIELD OF THE INVENTION

The present invention is related to a flame lamp, particularly to a flame lamp capable of simulating a flame effect and evaporating a fragrance.

### BACKGROUND

Lamp is a necessity in people's lives. With the improvement of living standards, the lamp is not only used for lighting, but also used as a decorative item in life. The beauty and atmosphere of the living space may be increased via the decorative lamp.

A flame lamp is a device capable of simulating a flame effect via LED components, which may control the color, swing and direction of the flame, and thereby simulate a visual feeling of flame shaking. The current flame lamp is only provided with the flame effect, and other special applications have not added therein, so that it' efficacy is monotonous.

### SUMMARY

It is one objective of the present invention to provide a flame lamp having fragrance, which comprises a top cover, a base, a lamp cover, and a control circuit. A tray is embedded in an upper surface of the top cover, and used for holding an essential oil. The lamp cover is disposed between the top cover and the base. The control circuit comprises a control board having a microprocessor, a flexible circuit board, and a heater. The flexible circuit board comprises a PWM driver and a plurality of LED lamp beads. The control board is disposed in the base. The flexible circuit board is disposed in the lamp cover. The heater is fixed on the bottom of the tray of the top cover. When the flame lamp operates, the microprocessor outputs a timing signal to the PWM driver. The PWM driver generates a PWM control signal according to the timing signal, and controls a blinking and light emitting intervals of the LED lamp beads via the PWM control signal, such that the LED lamp beads can simulate a flame effect. Besides, the heater heats the essential oil in the tray to evaporate a fragrance. Accordingly, the flame lamp can not only simulate a visual feeling of flame shaking, but also provide the fragrance, so that a romantic atmosphere can be created, and therefore user can relax heart and soothe emotion.

To achieve the above objective a flame lamp having fragrance, comprising: a top cover provided at an upper surface thereof with a tray used for holding an essential oil; a base; a lamp cover, being a transparent shell, and disposed between the top cover and the base; and a control circuit comprising a control board, a flexible circuit board, and a heater, wherein the control board comprises a microprocessor, the flexible circuit board comprises a PWM driver and a plurality of LED lamp beads, the control board is disposed in the base, the flexible circuit board is disposed in the lamp cover, the heater is disposed below a bottom of the tray, the microprocessor is connected to the PWM driver and the heater, and the PWM driver is connected to the plurality of LED lamp beads; when the microprocessor sends a timing signal to the PWM driver, the PWM driver generates a PWM control signal according to the timing signal, the PWM driver controls a blinking and light emitting intervals of the plurality of LED lamp beads via the PWM control signal to simulate a flame effect; wherein the heater heats the essential oil in the tray.

In one embodiment of the present invention, wherein the heater is a ceramic heater with a thermal resistor.

In one embodiment of the present invention, wherein the heater is fixed on the bottom of the tray of the top cover via a support member.

In one embodiment of the present invention, wherein the base is provided with an opening and a power switch, the control board further comprises a switch and a power supply interface, when the control board has disposed in the base, the switch of the control board will be next to the power switch, and the power supply interface will be located in the opening.

In one embodiment of the present invention, wherein the power supply interface is a DC interface or a USB interface.

In one embodiment of the present invention, wherein the microprocessor is connected to the power supply interface via the switch, when the switch is turned on by pressing the power switch, the power supply interface inputs a power supply to the control board via the switch, the microprocessor of the control board converts the power supply to a working voltage by a power converter, and provides the working voltage to the PWM driver and the heater.

In one embodiment of the present invention, wherein the flexible circuit board is fixed on the control board by means of welding points.

In one embodiment of the present invention, wherein the top cover is provided at a side of a lower edge thereof with a plurality of first engaging members, the lamp cover is provided at an upper inner edge thereof with a plurality of second engaging members corresponding to the plurality of first engaging members, the top cover is fixed on an upper end of the lamp cover by an engagement between the plurality of first engaging members and the plurality of second engaging members.

In one embodiment of the present invention, wherein the lamp cover is provided at the lower edge thereof with an annular groove, and a ring-shaped member is put in the annular groove.

In one embodiment of the present invention, wherein the microprocessor is connected to the heater by a 2P terminal connection wire.

### BRIEF DESCRIPTION OF THE DRAWINGS

FIG. 1 is a front view of a flame lamp of the present invention.

FIG. 2 is a rear view of the flame lamp of the present invention.

FIG. 3 is a top view of the flame lamp of the present invention.

FIG. 4 is a stereoscopic exploded view of the flame lamp of the present invention.

FIG. 5 is a plan exploded view of the flame lamp of the present invention.

FIG. 6 is a circuit diagram of the control circuit of the present invention

FIG. 7 is a circuit diagram of the flexible circuit board of the present invention.

### DETAILED DESCRIPTION OF THE PREFERRED EMBODIMENTS

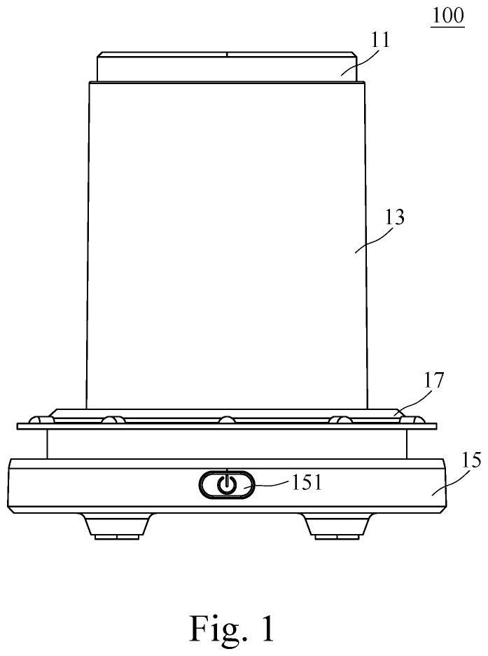

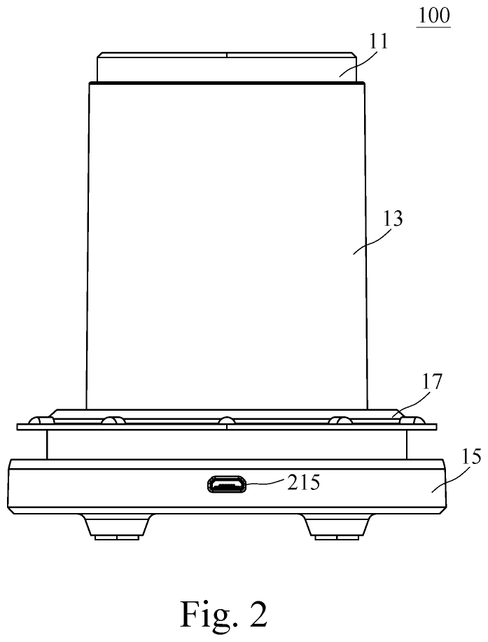

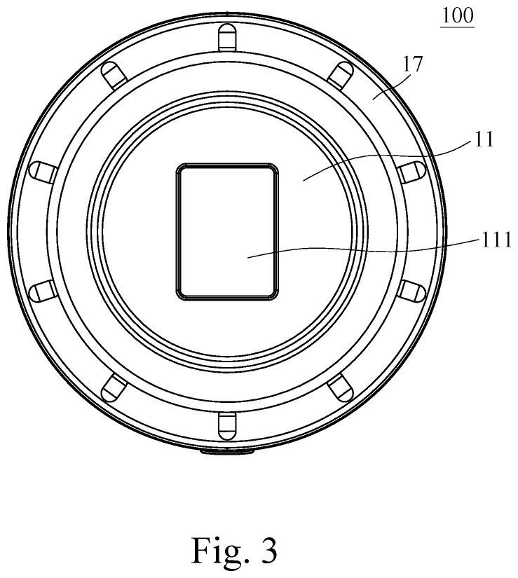

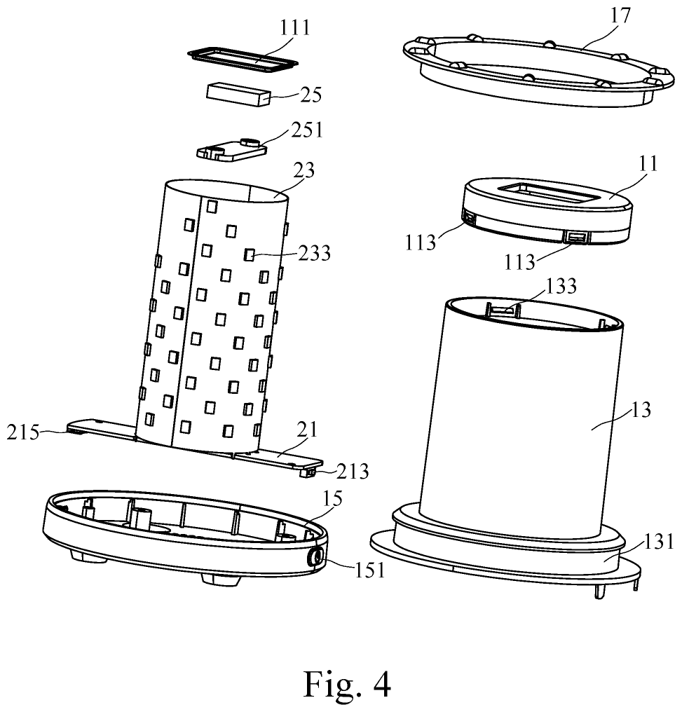

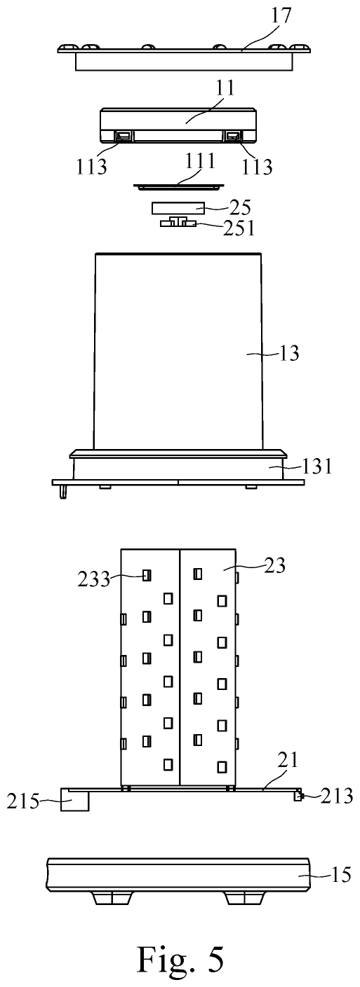

Referring to FIG. 1, FIG. 2, FIG. 3, FIG. 4, and FIG. 5, there are shown a front view, a rear view, a top view, a stereoscopic exploded view, and a plan exploded view of a flame lamp of the present invention. As shown in FIGS. 1 to 5, the flame lamp 100 comprises a top cover 11, a lamp cover 13, and a base 15. The lamp cover 13 is a transparent hollow round shell. A tray 111 is embedded in an upper surface of the top cover 11, and used for holding an essential oil. The top cover 11 is provided at the side of the lower edge thereof with a plurality of first engaging members 113, and the lamp cover 13 is provided at the upper inner edge thereof with a plurality of second engaging members 133 corresponding to the first engaging members 113. The top cover 11 is fixed on the upper end of the lamp cover 13 by an engagement between the first engaging members 113 and the second engaging members 133, for example, an engagement between convex members and concave members. The lamp cover 13 is provided at the lower edge thereof with an annular groove 131. A ring-shaped member 17 is put in the annular groove 131. The bottom of the lamp cover 13 is configured on the base 15 in a manner of embedding or locking. For example, the lamp cover 13 is provided at the bottom thereof with a plurality of insert members, the base 15 is provided with a plurality of connection members, the insert members of the lamp cover 13 insert into the connection members of the base 15 such that the lamp cover 13 can be fixed on the base 15; otherwise, the lamp cover 13 is provided at the bottom thereof with a plurality of holes, the base 15 is provided with a plurality of screw seats, screw members pass through the holes of the lamp cover 13, and then are locked on the screw seats of the base 15 such that the lamp cover 13 can be fixed on the base 15.

The control circuit 200 of the flame lamp 100 comprises a control board 21, a flexible circuit board 23, and a heater 25. The control board 21 is disposed in the base 15. The flexible circuit board 23 is disposed in the lamp cover 13 in a reeled form, and fixed on the control board 21 by means of welding points. The heater 25 is fixed on the bottom of the tray 111 of the top cover 11 via a support member 251. The heater 25 may be a ceramic heater with a thermal resistor.

The base 15 is provided with a power switch 151 at the periphery thereof. The control board 21 comprises a switch 213 and a power supply interface 215. When the control board 21 has disposed in the base 15, the switch 213 of the control board 21 will be next to the power switch 151. The base 15 is further provided with an opening at the periphery thereof. The power supply interface 215 will be located in the opening of the base 15. The power supply interface 215 is a DC interface, a USB interface or other interface capable of transmitting power energy. A DC adapter or a USB power cable can be connected to the power supply interface 215 to provide a power supply to the control board 21.

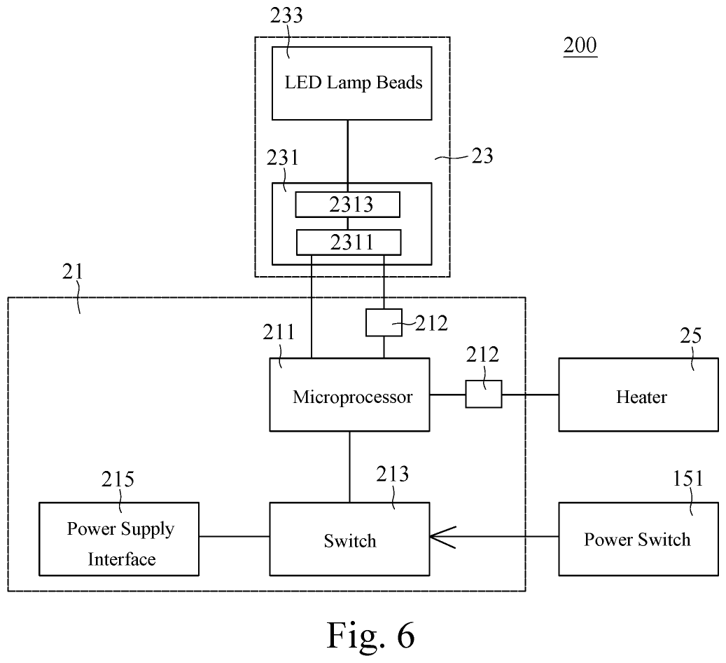

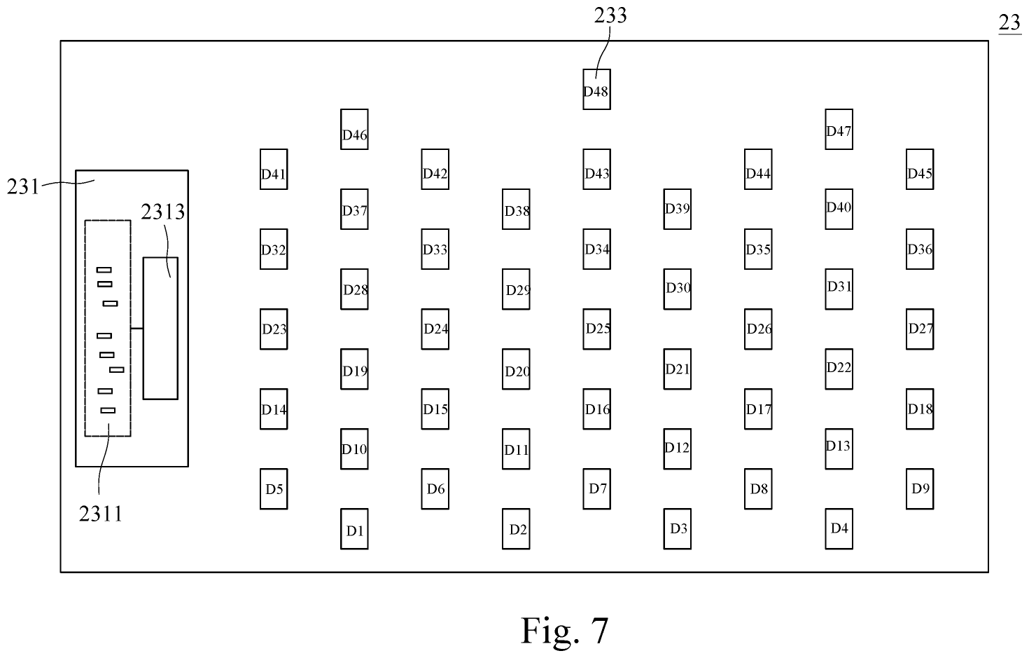

Referring to FIG. 6 and FIG. 7, there are shown a circuit diagram of the control circuit of the present invention and a circuit diagram of the flexible circuit board of the present invention. As shown in FIG. 6 and FIG. 7, the control board 21 further comprises microprocessor (such as MCU IC SOP-8) 211. The microprocessor 211 is connected to the power supply interface 215 via the switch 213. The flexible circuit board 23 comprises a PWM driver 231 and a plurality of LED lamp beads 233. The PWM driver 231 comprises a PWM modulation circuit 2311 and a control chip (such as MCU IC SOP-14) 2313. The PWM modulation circuit 2311 is connected to the PWM modulation circuit 2311 and a control chip 2313, and the control chip 2313 is further connected to the LED lamp beads 233. Besides, the microprocessor 211 is connected to the heater 25 via a 2 pin terminal connection wire.

When the power switch 151 is pressed, the switch 213 of the control board 21 is turned on, and the power supply is inputted to the control board 21 via the power supply interface 215. After the microprocessor 211 receives the power supply, the power supply will be converted into a working voltage by a power converter 212. For example, the power converter 212 converts a DC 12V power supply into a DC 5V working voltage and supplies the working voltage to the flexible circuit board 23 and the heater 25 such that the flexible circuit board 23 and the heater 25 can operate according to a power energy provided by the working voltage. In the present invention, the power converter 212 is consisted of an NPN transistor and a resistance component.

When the lamp 100 is operating, the microprocessor 211 sends a timing signal to the PWM driver 231. The PWM driver 231 generates a PWM control signal according to the timing signal. The PWM driver 231 controls a blinking and light emitting intervals of the LED lamp beads 233 via the PWM control signal.

One embodiment of the present invention, the flexible circuit board 23 includes 48 LED lamp beads, dimensions of which is 2835. In one embodiment of the present invention, these LED lamp beads 233 are arranged in 12 groups from bottom to top of the flexible circuit board 23. The 1st group (D1˜D4) and the 2nd group (D5˜D9) of the LED lamp beads 233 may be as a root part of the flame. The 3rd group (D10˜D13), the 4th group (D14˜D18), and the 5th group (D19˜D22) of the LED lamp beads 233 may be as a middle and lower part of the flame. The 6rd group (D23˜D27), the 7th group (D28˜D31), the 8th group (D32˜D36), and the 9th group (D37˜D40) of the LED lamp beads 233 may be as a middle and upper part of the flame. The 10rd group (D41˜D45), the 11th group (D46˜D47), and the 12th group (D48) of the LED lamp beads 233 may be as a top part of the flame.

The PWM control signal controls the blinking and light emitting intervals of each group of the LED lamp beads 233 in timing. For example, the PWM control signal controls that the 1st group (D1˜D4) and the 2nd group (D5˜D9) of the LED lamp beads 233 can light on at all times; the PWM control signal controls that the 3rd group (D10˜D13), the 4th group (D14˜D18), and the 5th group (D19˜D22) of the LED lamp beads 233 can blink every 24 ms; the PWM control signal controls that the 6rd group (D23˜D27), the 7th group (D28˜D31), the 8th group (D32˜D36), and the 9th group (D37˜D40) of the LED lamp beads 233 can blink every 40 ms; the PWM control signal controls that the 10rd group (D41˜D45), the 11th group (D46˜D47), and the 12th group (D48) of the LED lamp beads 233 can blink cyclically every 27 ms, 93 ms, 146 ms, and 255 ms. The blinking and light emitting intervals of each group of the LED lamp beads 233 can be controlled in the manner of timing, such that the flame lamp 100 can display a visual feeling of flame shaking.

Further, the heater 25 can execute to operate via the working voltage provided by the control board 21. When the heater 25 operates, it will heat the essential oil in the tray 111 to make that the essential oil heated can evaporate a fragrance. Besides, a time and temperature of heating of the heater 25 can be controlled by the microprocessor 211 to avoid excessive consumption of the essential oil in the tray 111.

Accordingly, the flame lamp 100 of the present invention can not only simulate a visual feeling of flame shaking, but also provide the fragrance, so that a romantic atmosphere can be created, and therefore user can relax heart and soothe emotion.

The above disclosure is only the preferred embodiment of the present invention, and not used for limiting the scope of the present invention. All equivalent variations and modifications on the basis of shapes, structures, features and spirits described in the claims of the present invention should be included in the claims of the present invention.

What is claimed is:

1. A flame lamp having fragrance, comprising:
a top cover provided at an upper surface thereof with a tray used for holding an essential oil;
a base;
a lamp cover, being a transparent shell, and disposed between the top cover and the base; and
a control circuit comprising a control board, a flexible circuit board, and a heater, wherein the control board comprises a microprocessor, the flexible circuit board comprises a PWM driver and a plurality of LED lamp beads, the control board is disposed in the base, the flexible circuit board is disposed in the lamp cover, the heater is disposed below a bottom of the tray, the microprocessor is connected to the PWM driver and the heater, and the PWM driver is connected to the plurality of LED lamp beads;
when the microprocessor sends a timing signal to the PWM driver, the PWM driver generates a PWM control signal according to the timing signal, the PWM driver controls a blinking and light emitting intervals of the plurality of LED lamp beads via the PWM control signal to simulate a flame effect;
wherein the heater heats the essential oil in the tray.

2. The flame lamp having fragrance according to claim 1, wherein the heater is a ceramic heater with a thermal resistor.

3. The flame lamp having fragrance according to claim 1, wherein the heater is fixed on the bottom of the tray of the top cover via a support member.

4. The flame lamp having fragrance according to claim 1, wherein the base is provided with an opening and a power switch, the control board further comprises a switch and a power supply interface, when the control board has disposed in the base, the switch of the control board will be next to the power switch, and the power supply interface will be located in the opening.

5. The flame lamp having fragrance according to claim 4, wherein the power supply interface is a DC interface or a USB interface.

6. The flame lamp having fragrance according to claim 4, wherein the microprocessor is connected to the power supply interface via the switch, when the switch is turned on by pressing the power switch, the power supply interface inputs a power supply to the control board via the switch, the microprocessor of the control board converts the power supply to a working voltage by a power converter, and provides the working voltage to the PWM driver and the heater.

7. The flame lamp having fragrance according to claim 1, wherein the flexible circuit board is fixed on the control board by means of welding points.

8. The flame lamp having fragrance according to claim 1, wherein the top cover is provided at a side of a lower edge thereof with a plurality of first engaging members, the lamp cover is provided at an upper inner edge thereof with a plurality of second engaging members corresponding to the plurality of first engaging members, the top cover is fixed on an upper end of the lamp cover by an engagement between the plurality of first engaging members and the plurality of second engaging members.

9. The flame lamp having fragrance according to claim 1, wherein the lamp cover is provided at the lower edge thereof with an annular groove, and a ring-shaped member is put in the annular groove.

10. The flame lamp having fragrance according to claim 1, wherein the microprocessor is connected to the heater by a 2P terminal connection wire.
